SEQUENCE LISTING (4 sequences)

SEQ ID NO: 1 - DNA, 23 bp, Artificial Sequence
  Description of Artificial Sequence  Primer
gcggatccatgggccaacaatcc

SEQ ID NO: 2 - DNA, 23 bp, Artificial Sequence
  Description of Artificial Sequence  Primer
gcaagcttttgctactttttagg

SEQ ID NO: 3 - DNA, 19 bp, Artificial Sequence
  Description of Artificial Sequence  Primer
ggcatcgtgatggactcgg

SEQ ID NO: 4 - DNA, 19 bp, Artificial Sequence
  Description of Artificial Sequence  Primer
gctggaaggtggacagcga

(19) United States
(12) Patent Application Publication
(10) Pub. No.: US 2006/0115817 A1
(43) Pub. Date: Jun. 1, 2006

(54) Span-xb gene and protein for the diagnosis and treatment of cancer

(76) Inventors: Seah H. Lim, Amarillo, TX (US); Zhiqing Wang, Amarillo, TX (US); Yana Zhang, Amarillo, TX (US)

Correspondence Address: Michael L Goldman;Nixon Peabody, Clinton Square, P O Box 31051, Rochester, NY, 14603, US

(21) Appl. No.: 10/525,623

(22) PCT Filed: Aug. 29, 2003
(86) PCT No.: PCT/US03/27338

§ 371 Date: Nov. 14, 2005

Related U.S. Application Data
(60) Provisional application No. 60/407,852, filed on Aug. 30, 2002.

(51) Int. Cl. C12Q 1/68; G01N 33/574

(52) U.S. Cl. 435/6; 435/7.23

(57) ABSTRACT

The present invention relates to methods for diagnosing cancer in a subject The method involves obtaining a biological sample from a subject and measuring SPAN-Xb expression level in the biological sample. The SPAN-Xb expression level in the biological sample is compared to the SPAN-Xb expression level in a control sample from a subject devoid of cancer, where a greater SPAN-Xb expression level in the biological sample compared to the SPAN-Xb expression level in the control sample is indicative of the subject having cancer. Methods for monitoring progression/regression of cancer in a subject are also disclosed. The present invention also relates to methods of treating a subject with cancer. The method involves administering to the subject a therapeutically effective amount of an agent capable of eliciting an immunological response against SPAN-Xb protein or specifically recognizing a SPAN-Xb protein, under conditions effective to-treat a cancer characterized by cells expressing SPAN-Xb.

This application claims the benefit of U.S. Provisional Patent Application Ser. No. 60/407,852, filed Aug. 30, 2002, which is hereby incorporated by reference in its entirety.

This invention arose out of research sponsored by the National Institutes of Health/National Cancer Institute (Grant No. RO1 CA88434). The U.S. Government may have certain rights in this invention.

### FIELD OF THE INVENTION

The present invention relates to the use of SPAN-Xb gene and protein for diagnosing, monitoring, and treating cancer in a subject.

### BACKGROUND OF THE INVENTION

Recent advances in tumor immunology suggest the potential immunogenicity of various tumors in the autologous setting. In addition, an array of antigenic targets on tumor cells has been defined using cytotoxic T-lymphocyte (CTL) clones against tumor cells, either through screening target cells transfected with recombinant tumor complementary DNA (cDNA) libraries (De Plaen et al., “Cloning of Genes Coding for Antigens Recognized by Cytolytic T Lymphocytes,” In: Lefkovits, The Immunology Methods Manual, ed., San Diego, Calif.: Academic Press, pp. 692-712 (1997)) or biochemical purification of peptides eluted from the major histocompatibility complex (MHC) molecules (Cox et al., “Identification of Peptide Recognized by Five Melanoma-specific Human Cytotoxic T Cell Lines,” Science 264:716-719 (1994)). More recently, Ling et al. (Ling et al., “Prevalence of Antibodies Against Proteins Derived from Leukemia Cells in Patients with Chronic Myeloid Leukemia,” Blood 92:4764-4770 (1998)) and others (Chen et al., “A Testicular Antigen Aberrantly Expressed in Human Cancers Detected by Autologous Antibody Screening,” Proc. Natl. Acad. Sci. USA 94:19141918 (1997); Sahin et al., “Human Neoplasms Elicit Multiple

Specific Immune Responses in Autologous Host,” Proc. Natl. Acad. Sci. USA, 92:11810-11813 (1995)) have shown that it is also possible to identify these novel tumor proteins without the need to establish tumor-reactive CTL clones using serologic screening of expression tumor cDNA library (SEREX). Unfortunately, all these methods are extremely labor intensive.

Through the technologies described above, it is apparent that neoplastic cells often aberrantly express normal testicular proteins. These proteins collectively form the new class of tumor antigens called cancer/testis antigens (CTAs). Because of the blood-testis barrier that limits contact between testicular and immune cells and the lack of human leukocyte antigen (HLA) class 1 expression on the surfaces of germ cells, the testis is an immune-privileged site. The restricted normal tissue distribution of CTAs to only the testis suggests that only tumor cells will be targeted during CTA immunotherapy; thus, these proteins are ideal candidates for tumor vaccines. Normal testicular proteins have often been found to be expressed at the mRNA level in hematologic malignancies (Lim et al., “Expression of Testicular Genes in Haematological Malignancies,” Br. J. Cancer 81:1162-1164 (1998); Lim et al., “Sperm Protein 17 is a Novel Cancer-Testis Antigen in Multiple Myeloma,” Blood 97:1508-1510 (2001)). In addition, Ling et al. (Ling et al., “Prevalence of Antibodies Against Proteins Derived from Leukemia Cells in Patients with Chronic Myeloid Leukemia,” Blood 92:4764-4770 (1998)) and others (Wu et al., “Detection of a Potent Humoral Response Associated with Immune-Induced Remission of Chronic Myelogenous Leukemia,” J. Clin. Invest. 160:705-714 (2000)) also found that patients with hematologic malignancies produced high-titer immunoglobulin G (IgG) responses against tumor-derived proteins, supporting their in vivo immunogenicity and their potential use as targets for immunotherapy of hematologic malignancies.

Through the use of bio-informatics involving an extensive search in the GenBank database for testicular-specific genes, followed by computer software prediction of the immunogenicity of the gene products, Sperm protein 17 (Sp17) has been identified as a novel CTA in multiple myeloma (MM) (Lim et al., “Sperm Protein 17 is a Novel Cancer-Testis Antigen in Multiple Myeloma,” Blood 97:1508-1510 (2001)) and ovarian cancer (Chiriva-Internati et al., “Tumor Vaccine of Ovarian Cancer Targeting Sperm Protein 17 (Sp 17),” Cancer 94:2447-2453 (2002)). HLA-class 1-restricted Sp17-specific CTLs that could lyse Sp17-positive MM (Chiriva-Intemati et al., “Sperm Protein 17 (Sp17) in Multiple Myeloma: Opportunity for Myeloma-Specific Donor T Cell Infusion to Enhance Graft-Versus-Myeloma Effect Without Increasing Graft-Versus-Host Disease Risk,” Eur. J. Immunol. 31:2277-2283.(2001); Chiriva-Intemati et al., “Successful Generation of Sperm Protein 17 (Sp17)-Specific Cytotoxic T Lymphocytes from Normal Donors: Implication for Tumor-Specific Adoptive Immunotherapy Following Allogeneic Stem Cell Transplantation for Sp17-Positive Multiple Myeloma,” Scand. J. Immunol. 56:429433 (2002); Chiriva-Intemati et al., “Sperm Protein 17 (Sp17) is a Suitable Target for Immunotherapy of Multiple Myeloma,” Blood 100:961-965 (2002)) and ovarian cancer cells (Chiriva-Internati et al., “Tumor Vaccine of Ovarian Cancer Targeting Sperm Protein 17 (Sp 17),” Cancer 94:2447-2453 (2002)) have also been successfully generated from healthy volunteers and patients with cancer.

Despite the advance in modern chemotherapy, the long term outlook of most cancer patients remain poor, suggesting the need for other therapeutic approaches. The above-mentioned immunotherapy/cancer vaccine is an ideal approach. Cancer vaccine is theoretically less toxic and more specific. In addition, the memory immune responses elicited by cancer vaccines may provide the long term cancer immune surveillance to prevent disease relapse. One of the major problems associated with the design of cancer vaccines is the paucity of suitable tumor antigens.

The present invention is directed to overcoming these deficiencies in the art.

### SUMMARY OF THE INVENTION

The present invention relates to a method for diagnosing cancer in a subject. The method involves obtaining a biological sample from a subject being tested and measuring SPAN-Xb expression level in the biological sample. The level of SPAN-Xb expression in the biological sample is then compared to the level of SPAN-Xb expression in a control sample from a subject who does not have cancer, where a greater level of SPAN-Xb expression in the biological sample compared to the level of SPAN-Xb expression in the control sample is indicative of the subject being tested having cancer.

Another aspect of the present invention relates to a method for monitoring cancer in a subject. The method involves obtaining a biological sample from a subject and measuring SPAN-Xb expression level in the biological sample. The level of SPAN-Xb expression in the biological sample is then compared to the level of SPAN-Xb expression in a control sample previously obtained from the subject, where an increase or decrease in the level of SPAN-Xb expression in the biological sample compared to the level of SPAN-Xb expression in the control sample is indicative of progression or regression of the cancer, respectively.

The present invention also relates to a method of treating a subject with cancer. The method involves administering to the subject a therapeutically effective amount of an agent capable of eliciting an immunological response against SPAN-Xb protein or specifically recognizing a SPAN-Xb protein, under conditions effective to treat a cancer characterized by cells expressing SPAN-Xb.

The present invention identifies SPAN-Xb, a testicular-specific protein, as a novel tumor antigen in hematologic malignancies and other solid tumors. The present invention demonstrates that SPAN-Xb gene expression is confined only to normal testis and not any other tissues, suggesting that immune responses generated against SPAN-Xb from a tumor vaccine will not likely affect any of the normal tissues. The present invention also demonstrates that SPAN-Xb (Westbrook et al., “Spermatid-Specific Expression of the Novel X-linked Gene Product SPAN-X localized to the Nucleus of Human Spermatozoa,” Biol. Reprod. 63:469-481 (2000), which is hereby incorporated by reference in its entirety), a spermatid-specific protein, is frequently expressed and elicits immune responses in vivo in patients with MM and other hematologic malignancies such as acute and chronic leukemias. The present invention provides the first demonstration of SPAN-Xb expression and in vivo immune responses to the protein in patients with MM and other hematologic malignancies. The restricted normal tissue expression of SPAN-Xb and its in vivo immunogenicity in cancer-bearing patients support the use of SPAN-Xb as a target for immunotherapy of SPAN-Xb positive tumors. Further, since its expression is seen only in tumors, SPAN-Xb could also be a marker for the diagnosis of those tumors.

### BRIEF DESCRIPTION OF THE FIGURES

FIG. 1 shows RT-PCR analysis of the SPAN-Xb gene in patients with hematologic malignancy using a pair of sequence-specific primers. SPAN-Xb mRNA could be detected only in patients with hematologic malignancy and not in the peripheral blood or bone marrow of healthy donors. Lane 1, peripheral blood from a healthy donor; lane 2, bone marrow from a healthy donor; lanes 3-6, tumor cells from patients with acute myeloid leukemia (AML) (lane 3), chronic lymphocytic leukemia (CLL) (lane 4), chronic myeloid leukemia (CML) (lane 5), and multiple myeloma (MM) (lane 6); lane 7, normal testis RNA; lane 8, positive control amplification using a plasmid containing SPAN-Xb cDNA. M indicates molecular marker; lane a, PCR of DNase I-treated RNA (i.e., PCR without RT); lane b, PCR of RNA that underwent RT; and lane c, control amplification for 13-actin gene fragment.

FIG. 2 shows restriction digest of the PCR products. Normal testis (lane 1), AML (lane 2), CML (lane 3), CLL (lane 4), and MM (lane 5) with BglI enzyme showing the specificity of the PCR products. M indicates molecular marker; a, mock digestion; and b, BglI digestion.

FIG. 3 depicts the RT-PCR analysis of RNA from a panel of normal tissue showing SPAN-Xb transcripts in only normal testis.

FIGS. 4A-B illustrate the successful generation of SPAN-Xb recombinant protein from E coli. FIG. 4A depicts Coomassie blue staining of a 12% SDS-polyacrylamide gel, under reduced conditions, showing purities of different aliquots of recombinant SPAN-Xb protein. FIG. 4B shows Western blot analysis of different aliquots of recombinant SPAN-Xb protein using anti-His tag antibodies, confirming the successful generation of recombinant SPAN-Xb protein. M indicates protein marker, lanes 1-2, recombinant protein from 2 different fractions of the eluates; lane 3, lysate of flow-through from washing; lane 4, lysate after passage through affinity column; lanes 5-6, different aliquots of recombinant E coli lysates; lane 7, lysate after 4 hours of protein induction; and lane 8, lysate at time 0 of protein induction.

FIGS. 5A-B depict ELISA analysis of diluted sera (1:1000) showing the presence of antibodies directed at SPAN-Xb in patients with hematologic malignancies but not in health donors. FIG. 5A shows ELISA for SPAN-Xb antibodies in patients with lymphoproliferative disorders (sample 1, mean±2 SD from 24 healthy donors; samples 2-45, MM; samples 46-57, CLL). FIG. 5B shows ELISA for SPAN-Xb antibodies in patients with myeloproliferative disorders (sample 1, mean±2 SD from 24 healthy donors; samples 2-21, CML; samples 22-23, AML). The bars with an asterisk depict samples with signals in excess of mean±2 SD from 24 healthy donors.

FIGS. 6A-B show Western blot analysis to determine the specificity of the anti-SPAN-Xb antibodies. FIG. 6A depicts Coomassie blue staining of a 12% SDS-polyacrylamide gel showing the loading of SPAN-Xb recombinant protein and a control recombinant protein Sp17 (M indicates protein marker; lane a, SPAN-Xb; and lane b, Sp17). FIG. 6B depicts Western blot analysis using diluted sera (1:1000) showing the presence of antibodies directed at SPAN-Xb and not Sp17 in patients with hematologic malignancies. Lane 1 indicates serum from patient with CML; lane 2, serum from patient with CLL; lane 3, serum from patient with AML; and lane 4, anti-6-His tag monoclonal antibodies. (Lane a, SPAN-Xb recombinant protein; lane b, Sp17 recombinant protein.)

### DETAILED DESCRIPTION OF THE INVENTION

The present invention relates to a method for diagnosing cancer in a subject. The method involves obtaining a biological sample from a subject being tested and measuring SPAN-Xb expression level in the biological sample. The level of SPAN-Xb expression in the biological sample is then compared to the level of SPAN-Xb expression in a control sample from a subject who does not have cancer, where a greater level of SPAN-Xb expression in the biological sample compared to the level of SPAN-Xb expression in the control sample is indicative of the subject being tested having cancer.

In one embodiment of the present invention, the SPAN-Xb expression level is measured by measuring the level of SPAN-Xb mRNA, using standard techniques known in the art such as reverse transcription-polymerase chain reaction (RT-PCR) or real-time quantitative PCR.

Alternatively, the SPAN-Xb expression level can be measured by measuring the level of SPAN-Xb protein. The SPAN-Xb protein level can be measured by standard assays known in the art, using an antibody recognizing a protein or polypeptide of SPAN-Xb. Examples of an assay system suitable for the determination of a SPAN-Xb antigen-antibody/binding portion complex include, without limitation, an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

The sequences for the SPAN-Xb gene and protein have been deposited and are disclosed in GenBank as accession number AF098307 (Westbrook et al., “Spermatid-Specific Expression of the Novel X-linked Gene Product SPAN-X localized to the Nucleus of Human Spermatozoa,” Biol. Reprod. 63:469-481 (2000), which is hereby incorporated by reference in its entirety).

Another aspect of the present invention relates to a method for monitoring cancer in a subject. The method involves obtaining a biological sample from a subject and measuring SPAN-Xb expression level in the biological sample. The level of SPAN-Xb expression in the biological sample is then compared to the level of SPAN-Xb expression in a control sample previously obtained from the subject, where an increase or decrease in the level of SPAN-Xb expression in the biological sample compared to the level of SPAN-Xb expression in the control sample is indicative of progression or regression of the cancer, respectively. If the subject received chemotherapy and/or radiation and/or surgery after the control sample was taken, a greater level of SPAN-Xb expression in the biological sample compared to the level of SPAN-Xb expression in the control sample would be indicative of the subject not responding to the treatment. In patients who have had elevated SPAN-Xb levels that resolved following treatment, a greater level of SPAN-Xb expression in the biological sample compared to the level of SPAN-Xb expression in the control sample would be indicative of the subject experiencing disease relapse.

The present invention also relates to a method of treating a subject with cancer. The method involves administering to the subject a therapeutically effective amount of an agent capable of eliciting an immunological response against SPAN-Xb protein or specifically recognizing a SPAN-Xb protein, under conditions effective to treat a cancer characterized by cells expressing SPAN-Xb.

In one embodiment of the present invention, the agent contains a recombinant SPAN-Xb protein capable of eliciting an immunological response against SPAN-Xb, thereby treating a cancer characterized by cells expressing SPAN-Xb.

In another embodiment of the present invention, the agent contains a nucleic acid molecule encoding SPAN-Xb. The SPAN-Xb nucleic acid molecule can be administered to the subject via delivery systems which are known to those of skill in the art of gene therapy. Exemplary delivery devices include, without limitation, liposomes, transdermal patches, implants, and syringes.

Gene therapy is a relatively new approach to treatment of diseases. Currently, gene therapy protocols relate to therapy of certain carefully chosen disorders, including certain inherited disorders, a number of aggressively fatal cancers, and AIDS (U.S. Pat. No. 6,316,416 to Patierno, which is hereby incorporated by reference in its entirety). The restricted application of gene therapy to a few disorders reflects concerns about the efficacy, safety, and ethical implications of the approach in general, and current techniques in particular. Despite the cautious approach mandated by these concerns, and despite the fact that techniques for carrying out gene therapy are still in an early stage of development, results from the first few trials have been very encouraging, some spectacularly so. It seems certain that gene therapy techniques will improve rapidly and that gene therapies soon will develop into an increasingly important and ubiquitous modality for treating disease (reviewed, for example, in Tolstoshev, Ann. Rev. Pharm. Toxicol. 32: 573-596 (1993) and Morgan et al., Ann. Rev. Biochem. 62:191-217 (1993), which are incorporated by reference herein in their entirety).

Gene therapy generally means the use of a nucleic acid molecule, in a cell, to achieve the production of an agent and the delivery of the agent to a cell or tissue, in situ, i.e., in a subject, to produce an anti-proliferative effect. Approaches to genetic therapy currently being developed, which can be used in accordance with this aspect of the present invention, often are grouped into two major categories: ex vivo and in vivo techniques.

Ex vivo techniques generally proceed by removing cells from a patient or from a donor, introducing a polynucleotide into the cells, usually selecting and growing out, to the extent possible, cells that have incorporated, and, most often, can express the polynucleotide, and then introducing the selected cells into the patient. Cells that target tumor cells in vivo, including tumor cells that have migrated from primary or secondary tumor sites, generally are preferred in this type of gene therapy.

In addition, as described further below, the nucleic acid molecule may be introduced directly into the subject. The nucleic acid in this case may be introduced systemically or by injection into a tumor site. The nucleic acid may be in the form of DNA or RNA, alone or in a complex, or in a vector.

The nucleic acid molecule may be in any of a variety of forms, for example, a DNA (in either a sense or antisense form), a DNA fragment cloned in a DNA vector, a DNA fragment cloned in DNA vector and encapsidated in a viral capsid, RNA, PNA, or other useful forms for introduction into the subject.

When incorporated into a vector, the nucleic acid construct may include a promoter, enhancer, and other cis-acting control regions that provide a desired level and specificity of expression in the cells of a region operably linked thereto that encodes an RNA, such as an anti-sense RNA, or a protein. The nucleic acid construct may contain several such operably linked control and encoding regions for expression of one or more mRNAs or proteins, or a mixture of the two.

The nucleic acid molecule encoding a SPAN-Xb protein or polypeptide may be introduced into cells either ex vivo or in vivo, including into a tumor in situ. A variety of techniques have been designed to deliver polynucleotides into cells for constitutive or inducible expression, and these routine techniques can be used in the gene therapy aspect of the present invention as well. Nucleic acid molecules will be delivered into cells ex vivo using cationic lipids, liposomes or viral vectors. Introduction into cells in vivo, including into cells of tumors in situ, will be using direct or systemic injection. Methods for introducing nucleic acid molecules in this manner can involve direct injection of a nucleotide, which then generally will be in a composition with a cationic lipid or other compound or compounds that facilitate direct uptake of DNA by cells in vivo. Such compositions may also comprise ingredients that modulate physiological persistence. In addition, the nucleic acid molecule can be introduced into cells in vivo in viral vectors.

Genetic therapies in accordance with the present invention may involve a transient (temporary) presence of the gene therapy polynucleotide in the patient or the permanent introduction of a polynucleotide into the patient. Genetic therapies, like the direct administration of agents discussed above, in accordance with the present invention may be used alone or in conjunction with other therapeutic modalities.

In another embodiment of the present invention, the agent contains a cytotoxic T-cell line which specifically recognizes a SPAN-Xb protein. A SPAN-Xb specific cytotoxic T-cell line capable of lysing autologous targets can be generated using dendritic cells to present the SPAN-Xb antigen. For methods of generating antigen-specific cytotoxic T-cell lines, see Lim et al., “Sperm Protein 17 is a Novel Cancer-Testis Antigen in Multiple Myeloma,” Blood 97:1508-1510 (2001), which is hereby incorporated by reference in its entirety.

Suitable methods of administrating the agent capable of eliciting an immunological response against SPAN-Xb protein or specifically recognizing a SPAN-Xb protein include orally, parenterally, subcutaneously, intravenously, intramuscularly, intraperitoneally, by intravesical instillation, by intracavitary, intravesical instillation, intraocularly, intraarterially, intralesionally, or by application to mucous membrane. Specifically, the method can be carried out subcutaneously, intravenously, or intramuscularly.

Examples of cancer that can be diagnosed or treated by the methods of the present invention include, but are not limited to, lung cancer, colon cancer, breast cancer, ovarian cancer, lymphoma, Hodgkin's disease, acute myeloid leukemia, chronic myeloid leukemia, acute lymphoid leukemia, chronic lymphoid leukemia, myeloma, and melanoma

The present invention is preferably directed to diagnosing, monitoring, and treating cancer in humans.

### EXAMPLES

The following examples are provided to illustrate embodiments of the present invention but are by no means intended to limit its scope.

### Example 1

### Materials

Clinical materials consisting of tumor cells, sera, and bone marrow were obtained from patients with hematologic malignancies or from healthy donors. All clinical materials were obtained after informed consent and with approval from the local ethics committee. Samples from presentation and relapse were included. Genomic DNA and RNA were prepared from these samples. All samples were cryopreserved at −80° C. until they were used in experiments.

### Example 2

### Reverse Transcription-Polymerase Chain Reaction

Total RNA was extracted from cells using the Tri-reagent (Sigma, St. Louis, Mo). All RNA specimens were first treated with DNase I (Ambion, Austin, Tex.) to remove genomic DNA contamination. First-strand cDNA was synthesized from 1 μg total RNA with random hexamer primers using the GeneAmp RNA PCR Core Kit (Roche Molecular Biochemicals, Indianapolis, Ind.) according to the manufacturer's recommendation. The polymerase chain reaction (PCR) primers were as follows: for 5′SPANXb PCR, 5′-GCG GAT CCA TGG GCC AAC AAT CC-3′ (SEQ ID NO: 1); for 3′ SPANXb PCR, 5′-GCA AGC TTT TGC TAC TTT TTA GG-3′ (SEQ ID NO: 2). They amplify a cDNA of 328 base pairs (bp). PCR was performed by means of 35 amplification cycles at an annealing temperature of 55° C. Positive control amplification contained a plasmid with the SPAN-Xb cDNA amplified from human testis RNA by reverse transcription (RT)-PCR and a negative control contained all the PCR reaction mixture except for substitution of cDNA by water. RNA integrity in each sample was checked by the amplification of a 615-bp β-actin gene segment with primers 5′-GGC ATC GTG ATG GAC TCG G-3′ (SEQ ID NO: 3) and 5′-GCT GGA AGG TGG ACA GCG A-3′ (SEQ ID NO: 4) at an annealing temperature of 67° C. Successful removal of genomic DNA contamination was confirmed in each sample by amplification of the RNA without RT reaction. PCR products were visualized on an ethidium bromide agarose gel for a DNA band of the expected size. All results were confirmed in 2 independent RT-PCRs. Specificity of all the PCR products was further confirmed by BglI restriction enzyme digest.

### Example 3

### Cloning and Sequence Analysis of SPAN-Xb

PCR products were cloned by the TA cloning system (Invitrogen, La Jolla, Calif.). Briefly, the PCR products were ligated into the pCR II TA cloning site that is flanked by 2 EcoRI restriction sites. Competent Escherichia coli (INV αF′ cells) were transformed by heat-shock method and plated for ampicillin and blue-white color selection on agar plates containing 5-bromo-6-chloro-3-indolyl-β-D-galactopyranoside. Recombinant plasmid DNA was extracted and purified for nucleotide sequence analysis by an automated DNA sequencer.

### Example 4

### Generation and Purification of SPAN-Xb Recombinant Protein

Full-length SPAN-Xb cDNA was isolated and amplified from normal testicular RNA by RT-PCR. Following gel purification of the PCR products, the cDNA was cloned into pQE30 vector (Qiagen, Valencia, Calif.) between the BamHI and HindIII sites to produce a recombinant fusion protein of SPAN-Xb that contained a 6-histidine (6-His) peptide at the N-terminal of the protein. This strategy allowed affinity purification of the recombinant protein in a nickel Sephadex column. In addition, the 6-His tag was chosen instead of other N-terminal tag because 6-His tag is generally poorly immunogenic; therefore, it would not be expected to contribute to the immunogenicity of the fusion protein. The recombinant plasmid was transformed into E coli and selected on agar plates for ampicillin resistance. The correct reading frame of SPAN-Xb cDNA in pQE30 was confirmed by DNA sequencing. To generate the recombinant protein, a recombinant clone was expanded in liquid culture and induced by 1 mM isopropyl P-D-thiogalactopyranoside (1PTG) for 4 hours. Recombinant SPAN-Xb protein was harvested from E coli lysate by sonication. Following passage through the Ni-NTA affinity column and numerous rounds of washing, the protein was eluted. Successful generation of recombinant SPAN-Xb protein was confirmed on sodium dodecyl sulfate-polyacrylamide gel electrophoresis (SDS-PAGE) by Coomassie blue staining and Western blot analysis using an antibody directed at the N-terminal 6-His tag (Qiagen).

### Example 5

### Enzyme-Linked Immunosorbent Assay

Antibodies directed at SPAN-Xb protein were detected in patient sera using an in-house (enzyme-linked immunosorbent assay) ELISA system. Briefly, 96-well flat-bottom microtiter plates were coated with the purified recombinant SPAN-Xb protein at a concentration of 5 μg/m. After 4-hour adherence of the antigen to the plate at 37° C., the wells were washed and then blocked with 3% bovine serum albumin in phosphate-buffered saline (PBS) at 37° C. for 2 hours. Patient sera were diluted 1:1000 with the blocking buffer and dispensed into the wells in triplicate and allowed to bind overnight at 4° C. Alkaline phosphatase-conjugated goat antihuman IgG (Sigma, St. Louis, Mo.) was then added to each well and incubated at room temperature for color development. Twenty-five microliters 2 N NaOH was added to stop the reaction. Color intensity was measured on a microplate reader (Molecular Devices, Sunnyvale, Calif.) and was analyzed using the Softmax data analysis program. In each experiment, 2 different controls were set up: one consisted of wells coated with a control E coli-derived recombinant 6-His fusion protein, and another consisted of wells coated with PBS before the addition of the blocking buffer. All results were confirmed in 2 independent experiments.

### Example 6

### Western Blot Analysis

Purified SPAN-Xb protein was fractionated in a 12% SDS-polyacrylamide gel and was transferred onto a nitrocellulose membrane. Successful generation of SPAN-Xb protein was confirmed using an antibody directed at the histidine tag. SPAN-Xb antibodies in the patient sera (1:1000 dilution) were detected by a goat antihuman IgG alkaline phosphatase conjugate. Antibody binding was visualized by reaction with the Western Blue-stabilized substrate (Promega, Madison, Mich.). An equal amount of another recombinant fusion protein derived from E coli was used as the negative control.

### Example 7

### SPAN-Xb Gene Expression in MM and Other Hematologic Malignancies

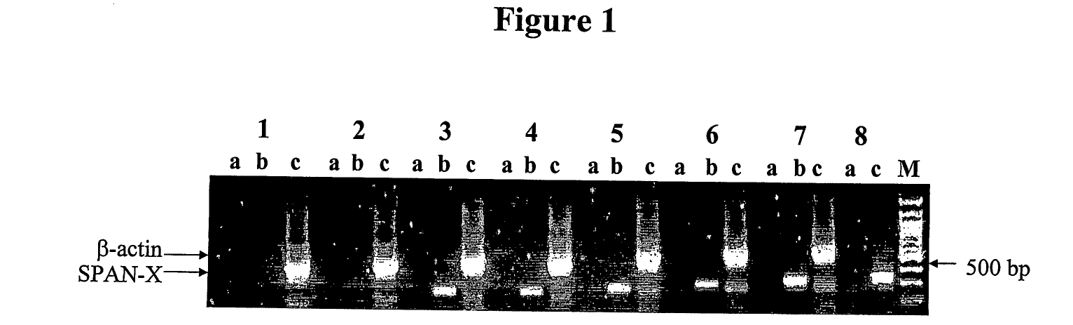

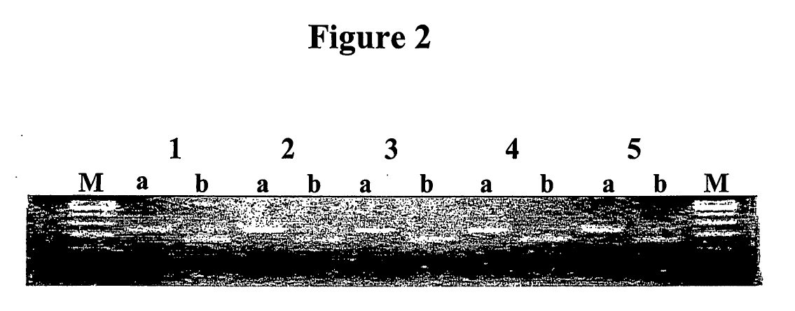

Using a pair of sequence-specific primers in RT-PCR, SPAN-Xb gene expression in tumor cells from patients with hematologic malignancies was first determined. SPAN-Xb transcripts could be detected in 6 (20%) of 30 patients with MM, 3 (33%) of 9 patients with chronic lymphocytic leukemia (CCL), 2 (29%) of 7 patients with chronic myeloid leukemia (CML), and 1 (50%) of 2 patients with acute myeloid leukemia (AML). In contrast, transcripts encoding SPAN-Xb could not be detected in any of the 20 peripheral blood samples or 14 bone marrow samples from health donors (P=0.001) (FIG. 1). SPAN-Xb gene expression was observed regardless of the sex of the patients. One PCR product from each disease type was randomly picked for sequence analysis. In all 4 disease types, nucleotide analysis confirmed SPAN-Xb sequence with no evidence of nucleotide or sequence mutation. Because there was a BglI internal restriction site within the SPAN-Xb cDNA but not within SPAN-Xa or CTp11, restriction digest with BglI was also used to determine whether the PCR products in all the positive specimens were in fact SPAN-Xb and not the closely related genes of SPAN-Xa and CTp11. Unlike testicular PCR products in which there was only partial digest of the DNA, suggesting the possible coamplification of SPAN-Xa or CTp11, all positive specimens completely digested with BglI enzymes (FIG. 2). These results, therefore, indicate the aberrant expression of SPAN-Xb in MM and other hematologic malignancies. In this sample of limited size, the expression of the SPAN-Xb gene did not appear to correlate with the stage of the disease because positive results were equally distributed between specimens obtained from patients in early- and late-stage disease. In addition, the SPAN-Xb gene was also equally distributed between patients with newly diagnosed disease and patients who experienced disease relapse following therapy.

The expression of Sp17 (Lim et al., “Sperm Protein 17 is a Novel Cancer-Testis Antigen in Multiple Myeloma,” Blood 97:1508-1510 (2001), which is hereby incorporated by reference in its entirety) and the lack of coexpression of Sp17 with another CTA, MAGE-Cl (Lim et al., “MAGE-CL (CT7) Gene Expression in Multiple Myeloma: Relationship to Sperm Protein 17,” Eur. J. Haematol, 67:332-334 (2001), which is hereby incorporated by reference in its entirety), in MM has previously been reported. Hence, it was next determined whether SPAN-Xb was coexpressed with Sp17 in MM, because CTA coexpression may provide the opportunity for the design of a polyvalent CTA vaccine for this disease. Using an RT-PCR method described in Lim et al. (Lim et al., “Sperm Protein 17 is a Novel Cancer-Testis Antigen in Multiple Myeloma,” Blood 97:1508-1510(2001), which is hereby incorporated by reference in its entirety), it was found that Sp17 was in fact coexpressed in 6 of 6 SPAN-Xb+samples from patients with MM. The expression of SPAN-Xb was, however, not solely linked to Sp17 because Sp17 transcripts were also detected in 3 of 20 SPAN-Xb- MM samples.

### Example 8

### SPAN-Xb is a CT Antigen

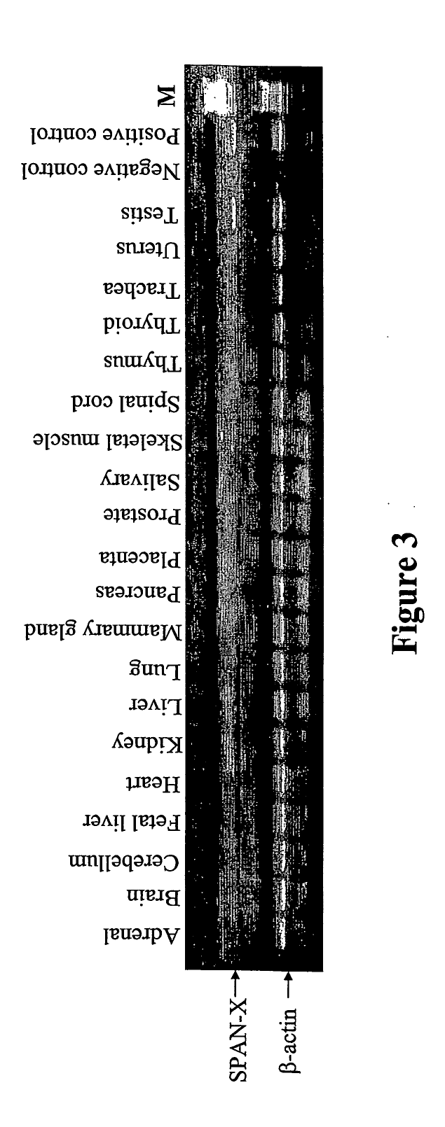

Because tissue specificity is a vital consideration in the choice of an antigen for immunotherapy, the expression of SPAN-Xb transcripts in total RNA derived from a panel of normal tissues was determined. By RT-PCR, it was demonstrated that SPAN-Xb mRNA showed restricted normal tissue expression and that the transcripts were detected only in normal testis (FIG. 3). These results confirm that SPAN-Xb is a CT antigen.

### Example 9

### Antibodies Against SPAN-Xb Can Be Detected in Patients with MM and Other Hematologic Malignancies

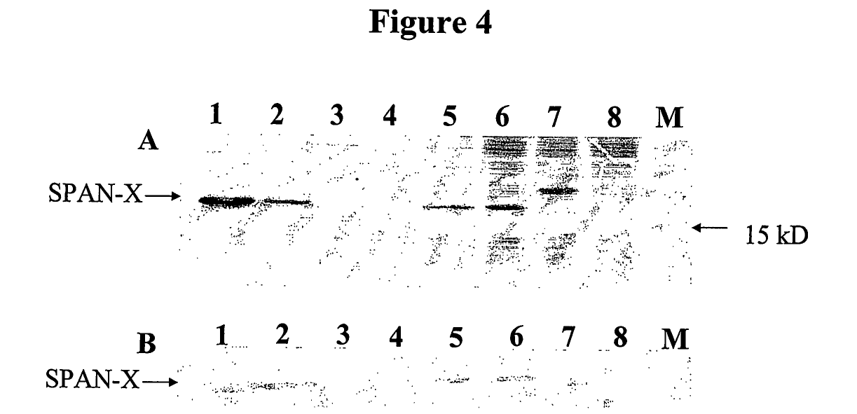

Although SPAN-Xb mRNA is frequently detected in patients with MM and other hematologic malignancies, it remained to be determined whether the mRNA was translated into SPAN-Xb protein that could be targeted by a tumor vaccine. An indirect method to support the in vivo translation of SPAN-Xb mRNA to protein is to determine whether antibodies against SPAN-Xb can be detected in these patients, because immune responses against a protein only occur after antigenic stimulation by the protein. To determine whether SPAN-Xb gene expression was associated with the development of immune responses against SPAN-Xb protein, a recombinant SPAN-Xb protein that would be suitable for use in ELISA and Western blot analysis was first generated. The full-length coding cDNA sequence of SPAN-Xb from normal testicular RNA was isolated and amplified and the recombinant protein was expressed in E coli as a fusion protein of SPAN-Xb with an N-terminal 6-His tag. After protein induction with isopropylthioglucose (IPTG), the recombinant protein was confirmed by SDS-PAGE followed by Coomassie blue staining and Western blotting. The recombinant protein was highly insoluble and was, therefore, dissolved in urea solution. Recombinant SPAN-Xb protein displayed a molecular size of approximately 20 kDa and was visualized in Western blot analysis by anti-His tag antibody (FIG. 4). Coomassie blue staining of the gel indicated that the purified recombinant protein was more than 95% pure (FIG. 4).

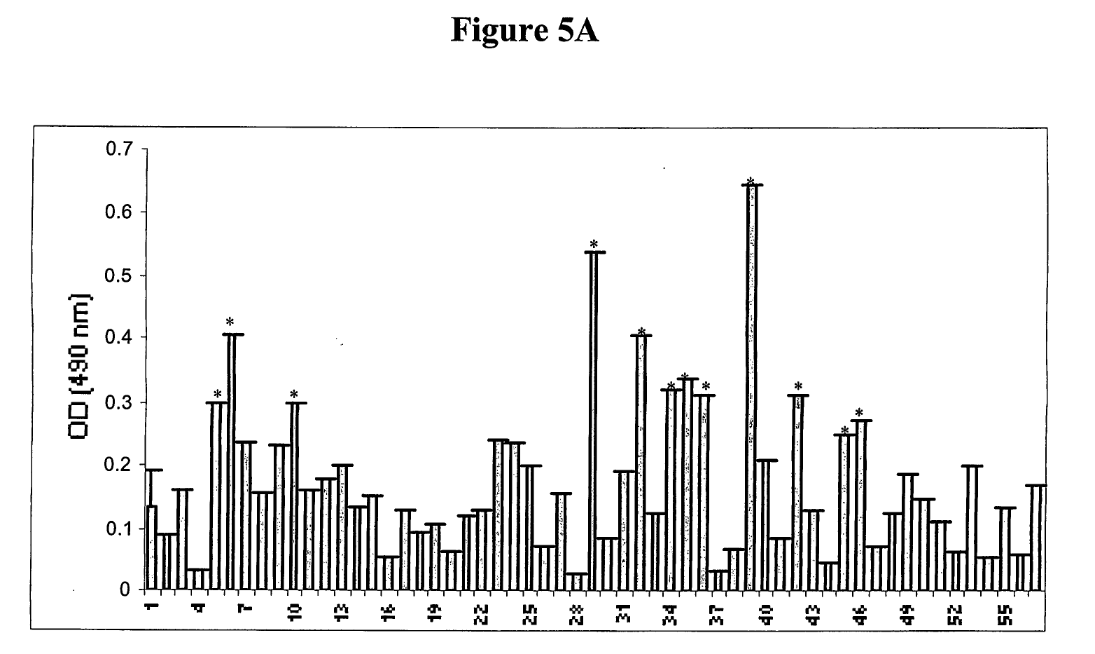

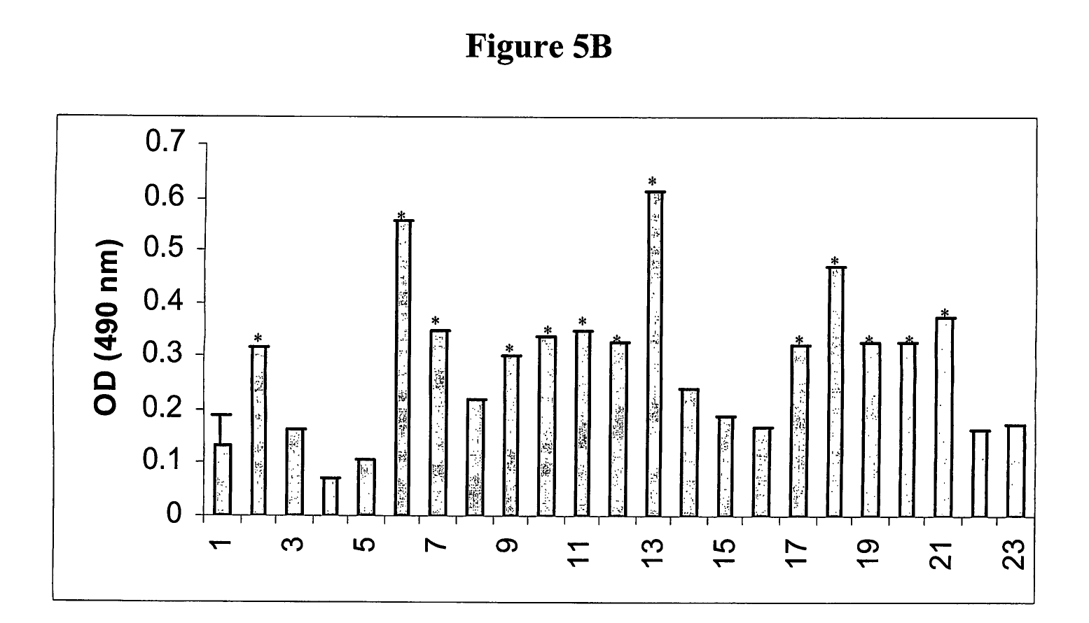

The purified SPAN-Xb protein was then used in an ELISA system to detect circulating antibodies against SPAN-Xb in the sera of patients with MM and other hematologic malignancies. The basal signals in the ELISA system were first established using sera from 24 healthy donors (mean±2 SD at OD₄₉₀ₘₘ=0.1325±0.059). Using the mean±2 SD from these 24 sera as the cutoff signal intensity at OD₄₉₀ₘₘ, it was found that high-titer IgG antibodies against SPAN-Xb protein were frequently detectable by ELISA in patients with MM and hematologic malignancies (FIGS. 5A-B). IgG antibodies against SPAN-Xb protein were detected by ELISA at a serum dilution of 1:1000 in 9 (20%) of 42 MM, 12 (60%) of 20 CML, 3 (33%) of 9 CLL, and 1 of 2 AML serum samples. These sera were tested in high dilution of 1:1000 to improve the specificity of the antibodies. In contrast, none of the sera from the 24 healthy donors were positive for SPAN-Xb antibodies. Binding of the antibodies from these sera to SPAN-Xb protein was specific, because no signal was observed in any of the samples in the 2 sets of control wells in the ELISA system (all showed signal intensities at OD₄₉₀ₘₘ of less than 0.010).

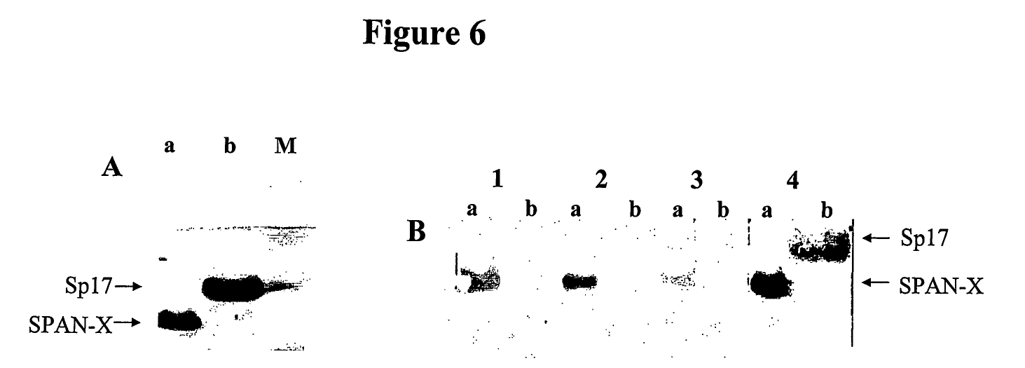

To further confirm the presence of SPAN-Xb antibodies, Western blot analysis was carried out applying these sera to the recombinant SPAN-Xb protein or to a control recombinant 6-His fusion protein also derived from E coli. Not surprisingly, because Western blot analysis is less sensitive than ELISA, not all ELISA-positive samples produced positive signals in Western blot analysis (Table 1; FIG. 6). However, all ELISA-negative samples produced negative signals in Western blot analysis. Based on the results of ELISA, the proportions of patients with detectable IgG antibodies against SPAN-Xb in these diseases correlated with the proportions of PCR-positive samples.

TABLE 1
Western Blot (WB) Analysis of Serum Samples

| Diagnosis, | | | | |
|---|---|---|---|---|
| no. | ELISA − | | | |
| analyzed | /WB⁺ | ELISA⁺/WB⁻ | ELISA⁻/WB⁺ | ELISA⁻/WB⁻ |
| AML, 1 | 1 | 0 | 0 | 0 |
| CLL, 5 | 2 | 0 | 0 | 3 |
| CML, 13 | 6 | 6 | 0 | 1 |
| MM, 7 | 3 | 4 | 0 | 0 |
| Donor, 6 | 0 | 0 | 0 | 6 |

Given that the results of restriction digest with BglI indicated the selective expression of SPAN-Xb and not SPAN-Xa or CTp11, the results support the in vivo immunogenicity of SPAN-Xb protein in patients with MM and other hematologic malignancies. Because SPAN-Xb mRNA was detected in these patients, the presence of IgG antibodies suggests the in vivo translation of SPAN-Xb mRNA into SPAN-Xb protein that was capable of eliciting B-cell responses in patients with cancer.

To determine the nature of the B-cell responses, the IgG subclass of the SPAN-Xb antibodies from 22 patients (7 MM, 12 DML, 2 CLL, and 1 AML) was determined using subclass-specific antihuman globulins in the ELISA system. It was found that SPAN-Xb antibodies were either of IgG2 or IgG3 subclass (Table 2). No IgG1 or IgG4 was detected.

TABLE 2
SPAN-Xb IgG Subclass

| Diagnosis, | | | | |
|---|---|---|---|---|
| no. analyzed | IgG1 | IgG2 | IgG3 | IgG4 |
| CML, 12 | 0 | 6 | 6 | 0 |
| MM, 7 | 0 | 4 | 3 | 0 |
| CLL, 2 | 0 | 1 | 1 | 0 |
| AML, 1 | 0 | 1 | 0 | 0 |

### Example 10

### Correlation Between Gene Expression and Immune Response

In 19 patients (2 AML, 9 CLL, 7 CML, and 1 MM), paired tumor samples and sera were available to determine the correlation between SPAN-Xb gene expression (GE) and B-cell immune response (IR) within individual patients. There was a close correlation between GE and IR, except in 2 patients with CML—one was GE+ without IR and the other was IR+ without GE (Table 3).

TABLE 3
Correlation Between GE and IR

| Diagnosis, | | | | |
|---|---|---|---|---|
| no. analyzed | GE⁻/IR⁺ | GE⁺/IR⁻ | GE⁻/IR⁺ | GE⁻/IR⁻ |
| AML, 2 | 1 | 0 | 0 | 1 |
| CLL, 9 | 2 | 1 | 0 | 6 |
| CML, 7 | 1 | 1 | 1 | 4 |
| MM, 1 | 0 | 0 | 0 | 1 |

As a result of the molecular events often associated with the malignant transformation of normal cells, neoantigens are produced from gene amplification, mutation, or the formation of fusion genes that arise from chromosomal translocation. These gene products are potentially immunogenic and may serve as targets of tumor vaccines. In addition to neoantigens, normal proteins that are aberrantly expressed in tumor cells are also candidates for immunotherapy. In particular, normal testicular proteins aberrantly expressed on tumor cells are excellent targets. As a result of their being from an immune-privileged site, these proteins are normally highly immunogenic, because the immune effector cells reactive against them are not subjected to thymic selection. Furthermore, because of the restricted normal tissue distribution, immunotherapy targeting testicular proteins is expected to be safe and without any nonspecific side effects. To isolate CTAs, various strategies have been investigated. They include applying patient sera to screen expression cDNA libraries constructed from either tumor RNA (Chen et al., “A Testicular Antigen Aberrantly Expressed in Human Cancers Detected by Autologous Antibody Screening,” Proc. Natl. Acad. Sci. USA 94:1914-1918 (1997), which is hereby incorporated by reference in its entirety) or normal testicular RNA (Jager et al., “Cancer-Testis Antigens and ING1 Tumor Suppressor Gene Product are Breast Cancer Antigens: Characterization of Tissue-Specific ING1 Transcripts and a Homologue Gene,” Cancer Res. 59:6917-6204 (1999), which is hereby incorporated by reference in its entirety). Identification and characterization of CTAs and immune responses against the proteins are needed.

Because of the extreme labor intensity of plaque screening in SEREX, an approach has recently been adopted that relies on screening the GenBank database for testicular-specific proteins and then using the available bio-informatics to predict the immunogenicity of these proteins in vivo. Through this approach, the present invention has identified a CTA, SPAN-Xb, for MM and other hematologic malignancies. The gene encoding SPAN-Xb is localized to Xq27.1 by fluorescence in situ hybridization (Westbrook et al., “Spermatid-Specific Expression of the Novel X-linked Gene Product SPAN-X localized to the Nucleus of Human Spermatozoa,” Biol. Reprod. 63:469-481 (2000), which is hereby incorporated by reference in its entirety), and the gene product is a spermatid-specific nuclear protein. It shows high degrees of homology at the nucleotide level (93%) and the protein level (86%) to CTp11, which is 98% and 97% homologous to the SPAN-Xa nucleotide and peptide sequences, respectively (Westbrook et al., “Differential Nuclear Localization of the Cancer/Testis-Associated Protein, SPAN-X/CTp11, in Transfected Cells and in 50% of Human Spermatozoa,” Biol. Reprod. 64:345-358 (2001), which is hereby incorporated by reference in its entirety). CTp11 is aberrantly expressed by cancer cells from patients with melanoma and with kidney, prostate, and bladder cancer (Zendman et al., “CTp11, a Novel Member of the Family of Human Cancer/Testis Antigen,” Cancer Res. 59:6223-6229 (1999), which is hereby incorporated by reference in its entirety). However, the expression of CTp11, SPAN-Xa, or SPAN-Xb in hematologic malignancies had never been investigated until the present invention. The results in the above examples indicate that SPAN-Xb transcripts are frequently aberrantly expressed in MM and other hematologic malignancies. The specificity of the findings was confirmed by nucleotide sequence analysis and restriction enzyme digest of the PCR products. Although the mechanisms leading to the aberrant expression of CTAs, such as SPAN-Xb, are unclear, it is likely a result of promoter demethylation that so often occurs in association with malignant cell transformation (Sigalotti et al., “Cancer Testis Antigen Expression in Mesothelioma: Role of DNA Methylation and Bioimmunotherapeutic Implications,” Br. J Cancer 86:979-982 (2002), which is hereby incorporated by reference in its entirety).

SPAN-Xb expectedly demonstrates a restricted normal tissue expression, as determined by RT-PCR analysis. Therefore, provided the transcripts encoding SPAN-Xb are translated to an immunogenic protein, SPAN-Xb could be a potential target for immunotherapy of these diseases. The work with Sp17 (Lim et al., “Sperm Protein 17 is a Novel Cancer-Testis Antigen in Multiple Myeloma,” Blood 97:1508-1510 (2001); Chiriva-Internati et al., “Tumor Vaccine of Ovarian Cancer Targeting Sperm Protein 17 (Sp 17),” Cancer 94:2447-2453 (2002); Chiriva-Intemati et al., “Sperm Protein 17 (Sp17) in Multiple Myeloma: Opportunity for Myeloma-Specific Donor T Cell Infusion to Enhance Graft-Versus-Myeloma Effect Without Increasing Graft-Versus-Host Disease Risk,” Eur. J Immunol. 31:2277-2283 (2001); Chiriva-Intemati et al., “Successful Generation of Sperm Protein 17 (Sp17)-Specific Cytotoxic T Lymphocytes from Normal Donors: Implication for Tumor-Specific Adoptive Immunotherapy Following Allogeneic Stem Cell Transplantation for Sp17-Positive Multiple Myeloma,” Scand. J Immunol. 56:429433 (2002); Chiriva-Internati et al., “Sperm Protein 17 (Sp17) is a Suitable Target for Immunotherapy of Multiple Myeloma,” Blood 100:961-965 (2002), which are hereby incorporated by reference in their entirety) illustrates the immunogenicity and translational potential of aberrantly expressed normal testicular proteins in these diseases.

Given that previous work by Ling et al. (Ling et al., “Prevalence of Antibodies Against Proteins Derived from Leukemia Cells in Patients with Chronic Myeloid Leukemia,” Blood 92:4764-4770 (1998), which is hereby incorporated by reference in its entirety) and others (Wu et al., “Detection of a Potent Humoral Response Associated with Immune-Induced Remission of Chronic Myelogenous Leukemia,” J. Clin. Invest. 160:705-714 (2000), which is hereby incorporated by reference in its entirety) demonstrated the ability of tumor-derived proteins to elicit B-cell immune responses in the autologous hosts in hematologic malignancies, it was determined whether high-titer IgG against SPAN-Xb could be detected in these patients. To do this, a SPAN-Xb recombinant protein was first generated from E coli. This was achieved through the cloning of the SPAN-Xb cDNA as a fusion gene to produce a recombinant SPAN-X protein that contained a 6-His tag at the N-terminal of the fusion protein. This strategy facilitated the purification of the recombinant protein by affinity column. The recombinant fusion protein from E coli was successfully generated. Surprisingly, the recombinant protein displayed on SDS-PAGE a molecular weight of approximately 20 kDa. This is identical to that obtained by an expression system using eukaryotic cells (Westbrook et al., “Differential Nuclear Localization of the Cancer/Testis-Associated Protein, SPAN-X/CTp11, in Transfected Cells and in 50% of Human Spermatozoa,” Biol. Reprod. 64:345-358 (2001), which is hereby incorporated by reference in its entirety), although the open reading frame (ORF) predicts the protein to be approximately 11 kDa. The discrepancy in the molecular weight of recombinant SPAN-Xb protein was previously attributed to a possible post-translational glycosylation of the protein (Westbrook et al., “Differential Nuclear Localization of the Cancer/Testis-Associated Protein, SPAN-X/CTp11, in Transfected Cells and in 50% of Human Spermatozoa,” Biol. Reprod. 64:345-358 (2001), which is hereby incorporated by reference in its entirety). However, the recombinant protein produced from a prokaryotic expression system was still larger than the predicted molecular weight, even though prokaryote-derived proteins are usually not glycosylated. Therefore, the apparently higher molecular weight of the protein on SDS-PAGE may be attributed to the poorly insoluble nature of the protein. SPAN-Xb recombinant protein is bacteria derived. To exclude immune responses caused by contaminating bacterial antigens that might have been present in the recombinant protein preparation, a control recombinant 6-His fusion protein, which was also prepared from the E coli lysate, was included in all experiments.

Using the recombinant protein in ELISA and Western blot analysis, B-cell responses, in the form of high-titer IgG, against SPAN-Xb protein occur frequently in patients with MM and other hematologic malignancies and not in healthy donors. Sequence analysis of the PCR products from these malignant cells did not show any mutation within the SPAN-Xb cDNA. Therefore, the immune responses were directed at the wild-type SPAN-Xb protein. Because immune responses against a protein only occur after exposure to the protein, SPAN-Xb transcripts expressed in tumor cells were likely translated to SPAN-X protein. Interestingly, the proportions of patients expressing the SPAN-Xb gene were nearly the same as the proportions of patients with detectable antibodies against SPAN-Xb. These results further support the notion that SPAN-Xb is not only expressed at the mRNA level but is also translated to its protein in vivo. In addition, they also support the in vivo immunogenicity of the protein in patients with cancer. Most important, because B-cell immune responses to an antigen, including tumor antigens such as NY-ESO-1, are usually generated only if the antigen is also immunogenic to T cells, the presence of high-titer IgG responses in these patients suggests that SPAN-Xb is potentially a target for T-cell-based immunotherapy of these malignant diseases.

The presence of in vivo immune responses against SPAN-Xb protein does not necessarily suggest antitumor effects. To further characterize the nature of the immune responses, the SPAN-Xb IgG subclasses in these patients were determined. Surprisingly, 2 distinct types of immune responses were observed: IgG2 antibodies suggestive of a Th2-type immune response and IgG3 antibodies suggestive of a Th 1 -type immune response. The distribution of the IgG subclass did not appear to be disease related because it was equally divided among patients of different disease types.

Although mRNA level generally correlates with protein level, exceptions frequently occur, ranging from total lack of translation, despite an abundance of mRNA, to high levels of protein without significant up-regulation of mRNA (Lim et al., “Defining Tumor Antigens: mRNA, Protein or Cytotoxicity?,” Trends Immunol. 23:236-237 (2002), which is hereby incorporated by reference in its entirety). Such discrepancies may explain the results that were obtained in one CML patient in whom the SPAN-Xb transcript was not detected by RT-PCR but in whom antibodies against SPAN-Xb protein were present. The close correlation observed between SPAN-Xb gene expression and immune responses suggests the in vivo translation of the SPAN-Xb gene in these diseases. Three of 4 patients with gene expression mounted antibody responses against SPAN-Xb protein. The ability to mount immune responses to an antigen depends on numerous factors, including the patient's age, HLA-type, and concurrent medications such as immunosuppressive agents. Therefore, any one of these factors could have led to the lack of immune responses observed in the one CML patient despite SPAN-Xb gene expression. In contrast, neither gene expression nor immune response was detected in any of the healthy donors.

In conclusion, SPAN-Xb is another novel CTA in MM and other hematologic malignancies. Its gene expression in these diseases is associated with the frequent development of antibody responses against SPAN-Xb protein. Although SPAN-Xb is a nuclear-associated protein and will not be amenable to targeting by monoclonal antibodies, SPAN-Xb could be a target for T cells because most high-titer IgG B-cell responses occur with cognitive help from T cells. Interestingly, despite the frequent presence of circulating high-titer IgG against SPAN-Xb, it has not been previously isolated using conventional SEREX approaches in CML (Ling et al., “Prevalence of Antibodies Against Proteins Derived from Leukemia Cells in Patients with Chronic Myeloid Leukemia,” Blood 92:4764-4770 (1998); Wu et al., “Detection of a Potent Humoral Response Associated with Immune-Induced Remission of Chronic Myelogenous Leukemia,” J. Clin. Invest. 160:705-714 (2000), which are hereby incorporated by reference in their entirety) or MM, suggesting that careful use of bio-informatics could complement the other antigen isolation methods currently used.

Although the invention has been described in detail, for the purpose of illustration, it is understood that such detail is for that purpose and variations can be made therein by those skilled in the art without departing from the spirit and scope of the invention which is defined by the following claims.

What is claimed:

1. A method for diagnosing cancer in a subject, said method comprising:
obtaining a biological sample from a subject being tested;
measuring SPAN-Xb expression level in the biological sample; and
comparing the level of SPAN-Xb expression in the biological sample to the level of SPAN-Xb expression in a control sample from a subject who does not have cancer, wherein a greater level of SPAN-Xb expression in the biological sample compared to the level of SPAN-Xb expression in the control sample is indicative of the subject being tested having cancer.

2. The method according to claim 1, wherein said measuring SPAN-Xb expression level comprises measuring the level of SPAN-Xb mRNA.

3. The method according to claim 1, wherein said measuring SPAN-Xb expression level comprises measuring the level of SPAN-Xb protein.

4. The method according to claim 3, wherein the level of SPAN-Xb protein is measured using an antibody recognizing the SPAN-Xb protein.

5. The method according to claim 1, wherein said cancer is selected from the group consisting of lung cancer, colon cancer, breast cancer, ovarian cancer, lymphoma, Hodgkin's disease, acute myeloid leukemia, chronic myeloid leukemia, acute lymphoid leukemia, chronic lymphoid leukemia, myeloma, and melanoma.

6. The method according to claim 1, wherein the subject is human.

7. A method for monitoring progression or regression of cancer in a subject, said method comprising:
obtaining a biological sample from a subject;
measuring SPAN-Xb expression level in the biological sample; and
comparing the level of SPAN-Xb expression in the biological sample to the level of SPAN-Xb expression in a control sample previously obtained from the subject, wherein an increase or decrease in the level of SPAN-Xb expression in the biological sample compared to the level of SPAN-Xb expression in the control sample is indicative of progression or regression of the cancer, respectively.

8. The method according to claim 7, wherein said measuring SPAN-Xb expression level comprises measuring the level of SPAN-Xb mRNA.

9. The method according to claim 7, wherein said measuring SPAN-Xb expression level comprises measuring the level of SPAN-Xb protein.

10. The method according to claim 9, wherein the level of SPAN-Xb protein is measured using an antibody recognizing the SPAN-Xb protein.

11. The method according to claim 7, wherein said cancer is selected from the group consisting of lung cancer, colon cancer, breast cancer, ovarian cancer, lymphoma, Hodgkin's disease, acute myeloid leukemia, chronic myeloid leukemia, acute lymphoid leukemia, chronic lymphoid leukemia, myeloma, and melanoma.

12. The method according to claim 7, wherein the subject is human.

13. A method of treating a subject with cancer comprising:
administering to the subject a therapeutically effective amount of an agent capable of eliciting an immunological response against SPAN-Xb protein or specifically recognizing a SPAN-Xb protein, under conditions effective to treat a cancer characterized by cells expressing SPAN-Xb.

14. The method according to claim 13, wherein said agent comprises a recombinant SPAN-Xb protein.

15. The method according to claim 13, wherein said agent comprises a nucleic acid molecule encoding SPAN-Xb.

16. The method according to claim 13, wherein said agent comprises a cytotoxic T-cell line which specifically recognizes a SPAN-Xb protein.

17. The method according to claim 13, wherein said cancer is selected from the group consisting of lung cancer, colon cancer, breast cancer, ovarian cancer, lymphoma, Hodgkin's disease, acute myeloid leukemia, chronic myeloid leukemia, acute lymphoid leukemia, chronic lymphoid leukemia, myeloma, and melanoma.

18. The method according to claim 13, wherein the subject is human.
